(19) United States
(12) Patent Application Publication
(10) Pub. No.: US 2022/0378715 A1
(43) Pub. Date: Dec. 1, 2022

(54) USE OF CANNABIDIOL PREPARATIONS IN THE TREATMENT OF ABSENCE EPILEPSY

(71) Applicant: GW Research Limited, Cambridge (GB)

(72) Inventors: Geoffrey GUY, Cambridge (GB); Volker KNAPPERTZ, Cambridge (GB); Benjamin WHALLEY, Cambridge (GB); Marie WOOLLEY-ROBERTS, Cambridge (GB)

(21) Appl. No.: 17/771,183

(22) PCT Filed: Oct. 23, 2020
(86) PCT No.: PCT/GB2020/052698

§ 371 Date: Apr. 22, 2022

(30) Foreign Application Priority Data
Oct. 25, 2019 (GB) 1915522.5

(51) Int. Cl. A61K 31/05; A61P 25/08

(52) U.S. Cl. CPC A61K 31/05; A61P 25/08

(57) ABSTRACT

The present invention relates to the use of a cannabidiol (CBD) preparation in the treatment of absence epilepsy. In particular the CBD preparation is characterized by chemical components and/or functional properties that distinguish them from prior CBD compositions. Preferably the CBD used is in the form of a botanically derived purified CBD which comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w). The botanically derived purified CBD preferably also comprises a mixture of both trans-THC and cis-THC. Alternatively, a synthetically produced CBD is used.

### FIELD OF THE INVENTION

The present invention relates to the use of a cannabidiol (CBD) preparation in the treatment of absence epilepsy. In particular the CBD preparation is characterized by chemical components and/or functional properties that distinguish them from prior CBD compositions.

Preferably the CBD used is in the form of a botanically derived purified CBD which comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w). The botanically derived purified CBD preferably also comprises a mixture of both trans-THC and cis-THC. Alternatively, a synthetically produced CBD is used.

### BACKGROUND TO THE INVENTION

Epilepsy is one of the most common neurological disorders, affecting approximately 1% (50-60 million people) of the worldwide population. Epilepsy is a common and debilitating disorder of the central nervous system defined as a brain state that promotes periodic unprovoked seizures, which are unpredictable and sometimes progressively severe.

Absence epilepsy is a generalized non-convulsive form of epilepsy, characterized by bilateral synchronous spike-wave discharges (SWDs) on the EEG associated with interruption of on-going activities lasting from a few seconds to half a minute, blank stare and possibly a brief upwards rotation of the eyes.

Absence seizures can be divided into two main forms: childhood absence epilepsy (CAE), with an age of onset of approximately 6 years, and juvenile absence epilepsy (JAE), with an age of onset of approximately 12 years. These idiopathic forms of generalized epilepsy affect 2%-10% of all paediatric epileptic patients. Furthermore, absence seizures can be also found in other epileptic syndromes such as Dravet syndrome, Panayiotopoulos syndrome, idiopathic occipital epilepsy, Gastaut-type idiopathic occipital epilepsy, West syndrome, Juvenile myoclonic epilepsy.

Treatment of absence epilepsy is generally started in childhood early after the identification of seizures. Fortunately, most children outgrow the seizures in adolescence, but a substantial minority continue to have seizures into adulthood and have significant long-term psychosocial sequela. Moreover, absence epilepsy has been recently linked with behavioural, affective and cognitive disturbances that may persist in adulthood. To date, co-morbidities are a rising problem in epilepsy and are frequently more harmful to patients than the seizures themselves.

Since comorbidity of epilepsy greatly diminishes the quality of life their treatment is a pressing need. In the past, neuropsychiatric disorders were considered as a consequence of the underlying seizure disorder. In contrast with this hypothesis, recently, new clinical and preclinical results have suggested the existence of a particular bidirectional link. In fact, it was demonstrated that not only patients with epilepsy have a greater risk to develop a neuropsychiatric disorder, but also patients with primary neuropsychiatric disorders are at greater risk of developing epilepsy.

This relationship also suggests the possible involvement of some common pathogenetic mechanisms to the onset of epilepsy and neuropsychiatric disorders such as depression, dementia and autism traits. Currently, patients with epilepsy are treated with pharmacological compounds (antiepileptic drugs: AEDs). These drugs are solely symptomatic therapies; which work to suppress seizures but do not possess antiepileptogenic or disease-modifying properties.

Moreover, the AEDs currently available have not improved the outcome of refractory epilepsy as well as the outcome of epileptic comorbidities, which are two of the major problems in epilepsy management.

Cannabidiol (CBD), a non-psychoactive derivative from the cannabis plant, has demonstrated anti-convulsant properties in several anecdotal reports, pre-clinical and clinical studies both in animal models and humans. Three randomized control trials showed efficacy of the purified pharmaceutical formulation of CBD in patients with Dravet and Lennox-Gastaut syndrome.

Based on these three trials, a botanically derived purified CBD preparation was approved by FDA in June 2018 for the treatment of seizures associated with Dravet and Lennox-Gastaut syndromes.

The present invention demonstrates an increased efficacy of a botanically derived purified CBD preparation which comprises minor amounts of the cannabinoids CBD-C1, CBDV, CBD-C4 and THC over a synthetic CBD which does not comprise minor amounts of cannabinoids in a rat model of absence seizures. These data are particularly surprising particularly given the fact that the concentration of CBD within the botanically derived purified CBD preparation and the synthetic preparation were the same.

### BRIEF SUMMARY OF THE DISCLOSURE

In accordance with a first aspect of the present invention there is provided a cannabidiol (CBD) preparation for use in the treatment of absence epilepsy.

Preferably the CBD preparation comprises greater than or equal to 98% (w/w) CBD and less than or equal to 2% (w/w) other cannabinoids, wherein the less than or equal to 2% (w/w) other cannabinoids comprise the cannabinoids tetrahydrocannabinol (THC); cannabidiol-C1 (CBD-C1); cannabidivarin (CBDV); and cannabidiol-C4 (CBD-C4), and wherein the THC is present as a mixture of trans-THC and cis-THC.

Preferably the CBD is present is isolated from cannabis plant material.

More preferably at least a portion of at least one of the cannabinoids present in the CBD preparation is isolated from cannabis plant material.

Alternatively, the CBD is present as a synthetic preparation. In a further embodiment at least a portion of at least one of the cannabinoids present in the CBD preparation is prepared synthetically.

Preferably the dose of CBD is greater than 5 mg/kg/day. More preferably the dose of CBD is 20 mg/kg/day. More preferably the dose of CBD is 25 mg/kg/day. More preferably the dose of CBD is 50 mg/kg/day.

In accordance with a second aspect of the present invention there is provided a method of treating absence epilepsy comprising administering a cannabidiol (CBD) preparation to the subject in need thereof.

### BRIEF DESCRIPTION OF THE DRAWINGS

FIG. 1 shows the drug product manufacturing process.

FIG. 2 shows the effects of a sub-chronic treatment with bCBD and sCBD on the established absence seizures.

FIG. 3 shows the effects of a sub-chronic treatment with bCBD and sCBD on the FST, EPM, PA and nORT tests.

FIG. 4 shows the effects of a sub-chronic treatment with bCBD and sCBD on the TCT.

### DEFINITIONS

Definitions of some of the terms used to describe the invention are detailed below:

Over 100 different cannabinoids have been identified, see for example, Handbook of Cannabis, Roger Pertwee, Chapter 1, pages 3 to 15. These cannabinoids can be split into different groups as follows: Phytocannabinoids; Endocannabinoids and Synthetic cannabinoids (which may be novel cannabinoids or synthetically produced phytocannabinoids or endocannabinoids).

“Phytocannabinoids” are cannabinoids that originate from nature and can be found in the cannabis plant. The phytocannabinoids can be isolated from plants to produce a highly purified extract or can be reproduced synthetically.

“Highly purified cannabinoids” are defined as cannabinoids that have been extracted from the cannabis plant and purified to the extent that other cannabinoids and non-cannabinoid components that are co-extracted with the cannabinoids have been removed, such that the highly purified cannabinoid is greater than or equal to 95% (w/w) pure.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form.

The human equivalent dose (HED) can be estimated using the following formula:

$$HED = Animal dose ( mg / kg ) multiplied by \frac{Animal Km}{Human Km}$$

The Km for a mouse is 3 and the Km for a human is 37.
Thus, for a human a 100 mg/kg dose in a mouse equates to a human dose of about 8.1 mg/kg.

### DETAILED DESCRIPTION

### Preparation of Botanically Derived Purified CBD

The following describes the production of the botanically derived purified CBD which comprises greater than or equal to 98% w/w CBD and less than or equal to other cannabinoids was used in Example 1 below.

In summary the drug substance used is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 95% CBD w/w, typically greater than 98% w/w.

The Cannabis sativa L. plants are grown, harvested, and processed to produce a botanical extract (intermediate) and then purified by crystallization to yield the CBD (botanically derived purified CBD).

The plant starting material is referred to as Botanical Raw Material (BRM); the botanical extract is the intermediate; and the active pharmaceutical ingredient (API) is CBD, the drug substance.

All parts of the process are controlled by specifications. The botanical raw material specification is described in Table A and the CBD API is described in Table B.

TABLE A
CBD botanical raw material specification

| Test | Method | Specification |
|---|---|---|
| Identification: | | |
| A | Visual | Complies |
| B | TLC | Corresponds to standard |
| | | (for CBD & CBDA) |
| C | HPLC/UV | Positive for CBDA |
| Assay: | In-house | NLT 90% of assayed |
| CBDA + CBD | (HPLC/UV) | cannabinoids by peak area |
| Loss on Drying | Ph. Eur. | NMT 15% |
| Aflatoxin | UKAS method | NMT 4 ppb |
| Microbial: | Ph. Eur. | |
| TVC | | NMT10⁷ cfu/g |
| Fungi | | NMT10⁵ cfu/g |
| E. coli | | NMT10² cfu/g |
| Foreign Matter: | Ph. Eur. | NMT 2% |
| Residual Herbicides | Ph. Eur. | Complies |
| and Pesticides | | |

TABLE B
Specification of an exemplary botanically
derived purified CBD preparation

| Test | Test Method | Limits |
|---|---|---|
| Appearance | Visual | Off-white/pale yellow crystals |
| Identification A | HPLC-UV | Retention time of major peak |
| | | corresponds to certified CBD |
| | | Reference Standard |
| Identification B | GC-FID/MS | Retention time and mass |
| | | spectrum of major peak |
| | | corresponds to certified |
| | | CBD Reference Standard |
| Identification C | FT-IR | Conforms to reference spectrum |
| | | for certified CBD Reference |
| | | Standard |
| Identification D | Melting Point | 65-67° C. |
| Identification E | Specific | Conforms with certified |
| | Optical | CBD Reference Standard; −110° |
| | Rotation | to −140° (in 95% ethanol) |
| Total Purity | Calculation | ≥98.0% |
| Chromatographic | HPLC-UV | ≥98.0% |
| Purity 1 | | |
| Chromatographic | GC-FID/MS | ≥98.0% |
| Purity 2 | | |
| CBDA | HPLC-UV | NMT 0.15% w/w |
| CBDV | | NMT 1.0% w/w |
| THC | | NMT 0.1% w/w |
| CBD-C4 | | NMT 0.5% w/w |
| Residual Solvents: | | |
| Alkane | GC | NMT 0.5% w/w |
| Ethanol | | NMT 0.5% w/w |
| Residual Water | Karl Fischer | NMT 1.0% w/w |

The purity of the botanically derived purified CBD preparation was greater than or equal to 98%. The botanically derived purified CBD includes THC and other cannabinoids, e.g., CBDA, CBDV, CBD-C1, and CBD-C4.

Distinct chemotypes of the Cannabis sativa L. plant have been produced to maximize the output of the specific chemical constituents, the cannabinoids. Certain chemovars produce predominantly CBD. Only the (−)-trans isomer of CBD is believed to occur naturally. During purification, the stereochemistry of CBD is not affected.

### Production of CBD Botanical Drug Substance

An overview of the steps to produce a botanical extract, the intermediate, are as follows:

a. Growing
b. Direct drying
c. Decarboxylation
d. Extraction—using liquid CO2
e. Winterization using ethanol
f. Filtration
g. Evaporation

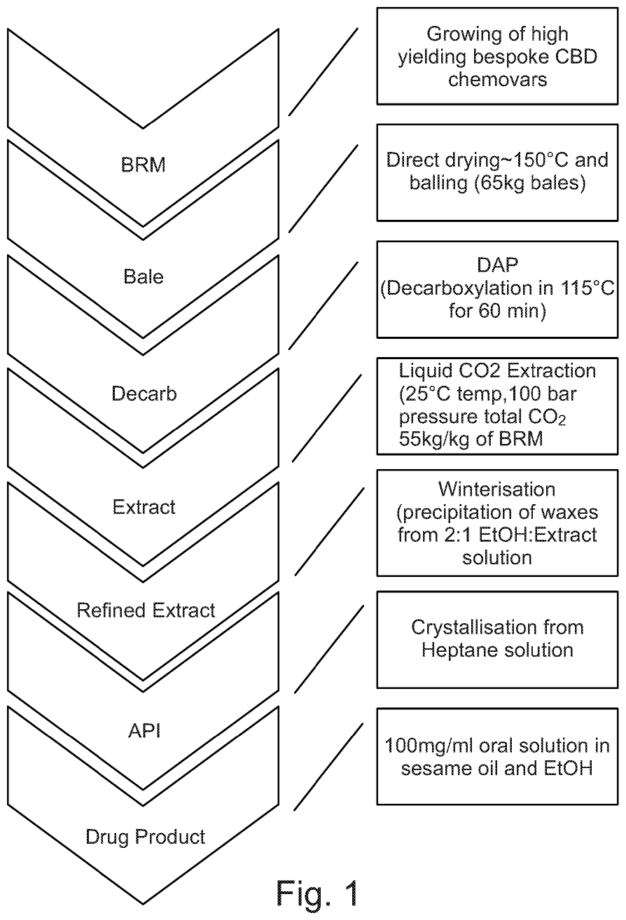

The process is described in greater detail below and in FIG. 1. High CBD yielding chemovars were grown, harvested, dried, baled and stored in a dry room until required. The botanical raw material (BRM) was finely chopped using an Apex mill fitted with a 1 mm screen. The milled BRM was stored in a freezer prior to extraction.

Decarboxylation of CBDA to CBD was carried out using heat. BRM was decarboxylated at 115° C. for 60 minutes.

Extraction was performed using liquid CO₂ at a temperature of 25° C. and 100 bar to produce botanical drug substance. The crude CBD BDS was winterized to refine the extract under standard conditions (2 volumes of ethanol at −20° C. for approximately 50 hours). The precipitated waxes were removed by filtration and the solvent was removed to yield the BDS.

Production of botanically derived purified CBD preparation

The manufacturing steps to produce the botanically derived purified CBD preparation from BDS were as follows:

a. Crystallization using C5-C12 straight chain or branched alkane
b. Filtration
c. Vacuum drying

The BDS produced using the methodology above was dispersed in C5-C12 straight chain or branched alkane. The mixture was manually agitated to break up any lumps and the sealed container then placed in a freezer for approximately 48 hours. The crystals were isolated via vacuum filtration, washed with aliquots of cold C5-C12 straight chain or branched alkane, in this case heptane, and dried under a vacuum of <10 mb at a temperature of 60° C. until dry. The botanically derived purified CBD preparation was stored in a freezer at −20° C. in a pharmaceutical grade stainless steel container, with FDA food grade approved silicone seal and clamps.

### Physicochemical Properties of the Botanically Derived Purified CBD

The botanically derived purified CBD used in the clinical trial described in the invention comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w).

The botanically derived purified CBD used additionally comprises a mixture of both trans-THC and cis-THC. It was found that the ratio of the trans-THC to cis-THC is altered and can be controlled by the processing and purification process, ranging from 3.3:1 (trans-THC:cis-THC) in its unrefined decarboxylated state to 0.8:1 (trans-THC:cis-THC) when highly purified.

Furthermore, the cis-THC found in botanically derived purified CBD is present as a mixture of both the (+)-cis-THC and the (−)-cis-THC isoforms.

Clearly a CBD preparation could be produced synthetically by producing a composition with duplicate components.

Example 1 below describes the use of a botanically derived purified CBD in a rat model of absence seizures.

### EXAMPLE 1

### Cannabidiol (CBD) in a Rat Model of Absence Seizures

This Example demonstrates the effect of botanically derived purified CBD and synthetic CBD in a rat model of absence seizures: Wistar Albino Glaxo/Rijswijk (WAG/Rij) rat model.

The WAG/Rij rat model is a well-established genetically determined model of absence seizures and epileptogenesis with neuropsychiatric-like comorbidities.

### Methods

### Test System

The effects of botanically derived purified CBD (>98%) (bCBD) and synthetic CBD (sCBD) at 10 and 100 mg/Kg, i.p. twice daily, for both drugs, was assessed in this animal model, by EEG-recordings and behavioural tests.

### Study Animals

Male Wistar Albino Glaxo/Rijswijk (WAG/Rij) rats at 1 month of age were obtained from Charles River Laboratories and used in this study.

In our animal house all rats were kept under the same environmental conditions (please, see below). The age range for rats subjected to EEG was P170-P190.

### Environmental Conditions

WAG/Rij rats were housed 3/4 per cage and kept under stable environmental conditions, humidity (60 ±5%), and temperature (21±2° C.), in a room with a 12/ 12-h reversed light/dark cycle (lights on at 20:00)

WAG/Rij rats were previously screened to assess their susceptibility to audiogenic seizures and only rats not susceptible to seizure were used in this study. Animal care and experimental procedures were conducted in conformity with the international and national law and policies (EU Directive 2010/63/EU for animal experiments, ARRIVE guidelines and the Basel declaration, including the B3R concept). The experimental protocols and the procedures reported herein were approved (Authorization n° 320/2019-PR) by the Animal Care Committee of the University of Catanzaro, Italy. All efforts were made to minimize animal suffering and to reduce the number of animals used.

### Drug Treatment Procedure

WAG/Rij rats were treated with bCBD or sCBD (10 and 100 mg/Kg, i.p. twice daily, for both drugs) over a chronic test period of at least 14 days.

Drug treatment was started at about 6 months of age (n=10 for every dose and drug group) and continued for 14 consecutive days, when experimental tests were performed (treatment was also continued during the experimental procedure phase. Age-matched control (vehicle) WAG/Rij rats (n =10) were kept under the same housing conditions over the same period of time with vehicle (1:1:18, Ethanol:kolliphor HS15:saline; i.p.).

WAG/Rij rats (n=10) underwent 2 EEG-recording periods in 2 consecutive days (one session per day). Every recording session lasted 3 hours and was performed 1 hour after drug administration.

To test the effects bCBD and sCBD on epileptic comorbidities, separate groups (n=10) of treated and vehicle WAG/Rij rats at 6 months of age were subjected to several behavioural tests (see below—cohort 2).

At the end of these experimental tests, treated and vehicle WAG/Rij rats were anesthetized and sacrificed to collect faecal, blood and brain samples 1-hour post-dose in the morning.

### Animal Surgery and Video-EEG Recording (Cohort 1)

WAG/Rij rats around the age of 6 months were chronically implanted, under anaesthesia using a Kopf stereotaxic instrument, with 3 cortical electrodes for EEG recordings. Stainless steel screw electrodes were implanted on the dura mater over the cortex: in the frontal region (AP=−1 mm;L=±2.5 mm) and in the parietal region (AP=−5 mm; L=±2.5 mm); the ground electrode was placed over the cerebellum. The animals were attached to a multichannel amplifier (PINNACLE TECHNOLOGY INC, 2721 OREGON ST., LAWRENCE, KANS. 66046) by a flexible recording cable and an electric swivel, fixed above the cages, permitting free movements for the animals.

All animals were allowed at least 1 week of recovery and handled twice a day. In order to habituate the animals to the recording conditions, rats were connected to the recording cables, for at least 3 days before the experiments. Every EEG recording was always performed starting at 9.00 am in order to avoid circadian alterations within groups. All EEG signals were amplified and conditioned by analogue filters (filtering: below 1 Hz and above 30 Hz at 6 dB/octave) and subjected to an analogue-to-digital conversion with a sampling rate of 300 Hz. The blinded quantification of absence seizures was based on the number and the duration of EEG Spike Wave Discharges (SWDs).

### Behavioural Tests (Cohort 2-4)

All behavioural tests were performed 1-hour post-dose in the morning. All experimental groups were subjected to a single test per day. Each rat started test 1-hour post-dose in the morning.

Regarding treated groups, when the same group of rats was subjected to multiple behavioural tests and/or repeated sessions of the same test, rats were kept under treatment. Furthermore, when 2 tests were performed on the same animal, at least 1 day (range 1-3 days) was allowed. Experiments were always performed between 09:00 and 12:00 in order to avoid possible circadian alteration of test results.

All behavioural tests, except for passive avoidance (PA) and rotarod, were performed with the support of video-tracking software (EthoVision XT8; Noldus Information Technology, Wageningen, the Netherlands).

### Forced Swimming Test (FST) (Cohort 4)

The FST, is currently the most reliable method to rapidly screen compounds for antidepressant activity in rodents; we used an FST protocol previously standardized in our laboratories for WAG/Rij rats.

Briefly, rats were placed individually for 6 min into a glass cylinder (height 47 cm, diameter 38 cm) filled with 38 cm of water maintained between 23° C. and 25° C. The total duration of immobility (immobility time) was recorded during the last 4 min of a 6-min testing period. The criterion for immobility and passive swimming (immobility time) was floating vertically in the water while making only those movements essential to keep the head above the surface of the water, which is directly proportional to depressive-like behaviour. At the end of the FST, rats were removed and dried with a towel before being returned to their home cage. Mean swimming velocity and total distance moved were also measured and examined for every experimental group in order to check for any obvious locomotor impairment.

### Elevated Plus Maze (EPM) (Cohort 3)

The EPM consists of 2 opposing open arms and 2 opposing closed arms of the same size (45 cm×10 cm) with 10 cm high walls connected by a central platform (10×10 cm) and elevated 80 cm above the floor. Rats are positioned in the central platform facing a closed arm and the number of entries into, time spent on each arm, and central platform are measured.

The maze was systematically cleaned (Disposable towels and 70% ethanol solution) to remove olfactory cues, after each animal was tested. The shorter the time spent in open arms and central platform the higher the anxiety is and vice versa. Mean velocity and total distance moved were also measured and examined for every experimental group.

### Passive Avoidance (PA) (Cohort 2)

Rats were tested in a step-through type passive avoidance apparatus (Ugo Basile, Italy, model 40,550), measuring 57×27×30 cm, consisting of a cage divided into two chambers (light and dark) by a sliding door.

The PA test was carried out over two successive days. Briefly, on day one (pre-acquisition trial or habituation), rats were introduced individually in the light compartment and allowed to freely explore the cage for 5 min. The acquisition or conditioning trial was conducted 15 min after habituation. Rats were individually introduced in the light compartment.; following a 30 s adaptation time, the sliding door between the two compartments was opened. When rats completely entered into the dark compartment, the sliding door was automatically closed and an electrical foot-shock (0.5 mA for 3 s) was delivered via the floor grid.

The latency to enter (s) in the dark compartment was detected and analysed. The retention or memory trial was evaluated 24 h after the acquisition trial, by recording their latency to enter (s) in the dark compartment. The latency to enter in the dark compartment was directly linked to the retention memory: the better the memory, the greater the latency.

Novel Object Recognition Test (nORT) (Cohort 2)

The nORT was performed as previously described by Bartolini et al. with some minor modifications. Briefly, three days before nORT, rats were exposed to a habituation trial lasting 6 min.

During this trial, each rat was allowed to freely explore an opaque open field Plexiglas box (70×70×30 cm) [32,44]. To investigate short-term memory, in the familiarization trial (T1) two identical objects (wood cubes) were located in two opposite corners of the box. Each rat was placed in the centre of the open field, facing away from the sample objects and left to explore freely the objects for a maximum of 5 min, before returning to its home cage. The familiarization trial ended when the rat completed 20 s of exploration for each object.

Exploration was considered directing the nose at a distance <2 cm to the object and/or touching it with the nose and sniffing. After a retention interval of 60 min the test phase (T2) was conducted. During this phase a novel object with different shape (wood pyramid) and colour (green) replaced one of the now familiar objects (yellow wood cube) displayed in TI and rats were left in the box for 5 min.

After this session, the rat returned to home cage. The time spent exploring the familiar (F) and the novel object (N) was recorded separately and a discrimination index (DI) was calculated (DI=N−F/N+F). To avoid olfactory cues both the objects and the box were cleaned with 70% ethanol solution after each session.

Furthermore, to avoid place preference, the role (familiar or novel object) and the position of the two objects during T2 were randomly changed. Total distance moved (cm) was also evaluated and analysed for every experimental group in order to check for locomotor deficits.

### Three Chamber Test (TCT) (Cohort 3)

Three-chambered approach (sociability) is an automated, widely used and well validated assay that compares time that the subject (rodent) spends with a novel animal versus time spent with a non-social inanimate object.

The three-chamber apparatus is a non-transparent white plastic box with two transparent acrylic partitions with a rectangular opening. The acrylic partitions divide the box into three chambers (left, centre, and right), illuminated at 120 lux. The animal is placed in the middle chamber with the dividers closed to allow it to explore the middle chamber for ten minutes.

After this habituation period, an unfamiliar adult animal is placed inside a small wire cage in one of the side chambers. An identical wire cage with an inanimate object is placed in the opposite chamber. The dividers are then raised, allowing the test subject to move freely throughout all three chambers of the apparatus over a 10-minute test session.

Sociability is defined as the time spent by the subject rat in the chamber containing the novel rat vs the time spent in the chamber containing the inanimate novel object. A second corroborative measure is the time spent sniffing the novel rat vs the time spent sniffing the novel object, which measures direct social interactions.

Number of transitions across chambers offers a built-in control measure of exploratory locomotion. Most strains of rodents spend more time with the novel animal versus the object representing normal sociability. Equal or less time spent with the novel animal would represent the absence of sociability in this task. Rodents investigate novel conspecifics by directed sniffing to the face, nose, anogenital or other body regions, depending on the relative positions of the two animals. The three-chambered test was carried out with the support of EthoVision, video tracking software, and equipment from Noldus (Wageningen, The Netherlands).

### Rotarod Test (RT) (Cohort 4)

The Rotarod apparatus (Ugo Basile, Varese, Italy) consists of a base platform and a rotating rod with a diameter of 6 cm and a non-slippery surface. The integrity of motor coordination is assessed on the basis of the time the animals keep their balance on the accelerating rotating rod up to a maximum of 10 min (600 s).

Briefly, rats were pre-trained for 2 days in order to reach a stable performance. After the training, the rotation of the rod was gradually accelerated from 5 rpm to 50 rpm. After a maximum of 3 falls from the rod, the test is suspended and the time is recorded, the result is a mean of the 3 measures.

### Statistical Analyses

All statistical procedures were performed using GraphPad Prism 6.0 (GraphPad Software, Inc., La Jolla, CA, USA). EEG recordings were subdivided into 30-min epochs, and the duration and number of SWDs were evaluated separately.

The results were tested for normality using the D′Agostino & Pearson test. If the data were normally distributed, a 1-way ANOVA was used. When a significant interaction between independent variables or a significant main effect of any of the independent variables was found, a Sidak's multiple comparison test was then performed. In the situation of non-normally distributed values, Kruskal-Wallis was used and if significant, planned multiple comparisons were performed using the Dunn's post-hoc test. A 2-way ANOVA was used to analyse the data obtained in the Three-Chamber social task.

### Results

### Effects of Treatments on Established Absence Seizures

Analysis of EEG recordings from control adult WAG/Rij rats at 6 months of age revealed that the mean number of spike wave discharges (nSWDs) for a 30-min epoch (i.e., SWDs per 3 hr/6) was 10.75±1.0205 with a mean total duration (dSWDs) of 62.2+7.18 s and a mean single duration (sSWD) of 5.78±0.85 s.

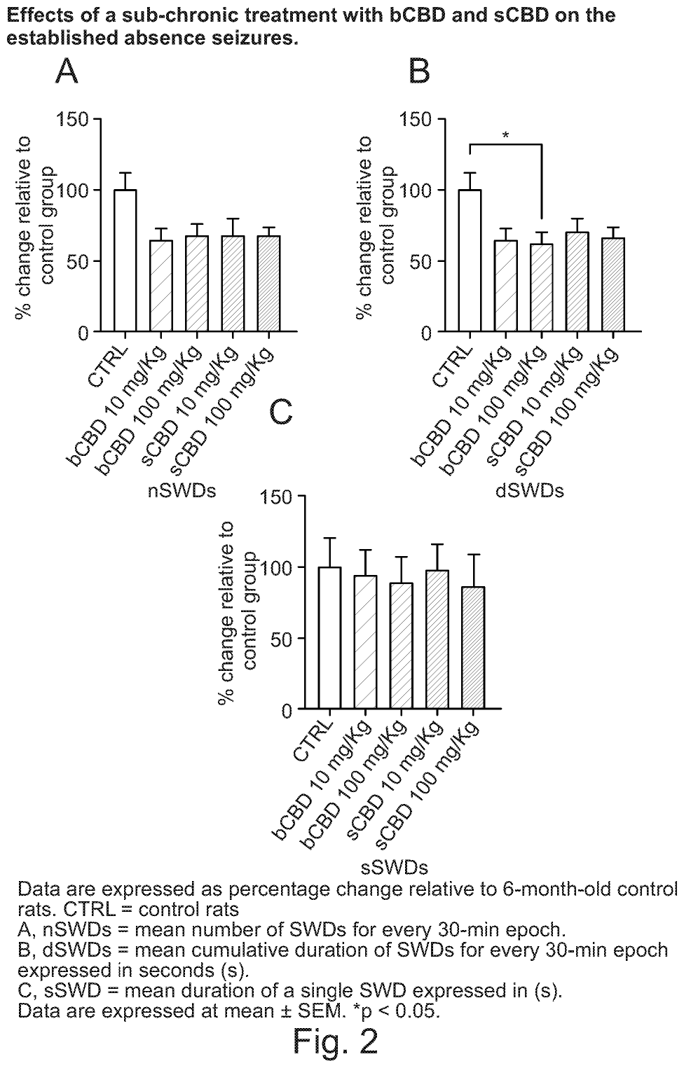

As shown in FIG. 2A, administration of bCBD and sCBD, in adult WAG/Rij rats, did not significantly (F4,45=2.572; p=0.0504; n=10 per group) alter the nSWDs in comparison to control (vehicle) group.

However, the dSWDs were significantly affected by the treatments (F4,45=2.752; p=0.039; n=10 per group) as is shown in FIG. 2B. The EEG recordings demonstrated that bCBD, at 100 mg/Kg, induced a significant reduction of dSWDs of about 38% (p=0.0388).

No significant effect of treatment on sSWDs was observed (F4,45=0.042; p=0.99; n=10 per group) as shown in FIG. 2C.

No differences were detected between both drugs, at the two doses used, on EEG parameters. Animal growth, over the 14 days of treatment, did not significantly differ between treated and vehicle rats (data not shown).

Behavioural tests

### Forced Swim Test (FST)

The FST was performed to evaluate depressive-like behaviour in both vehicle and treated WAG/Rij, which, from the fourth month onwards, exhibit an increased immobility time in the FST in comparison to age matched Wistar rats.

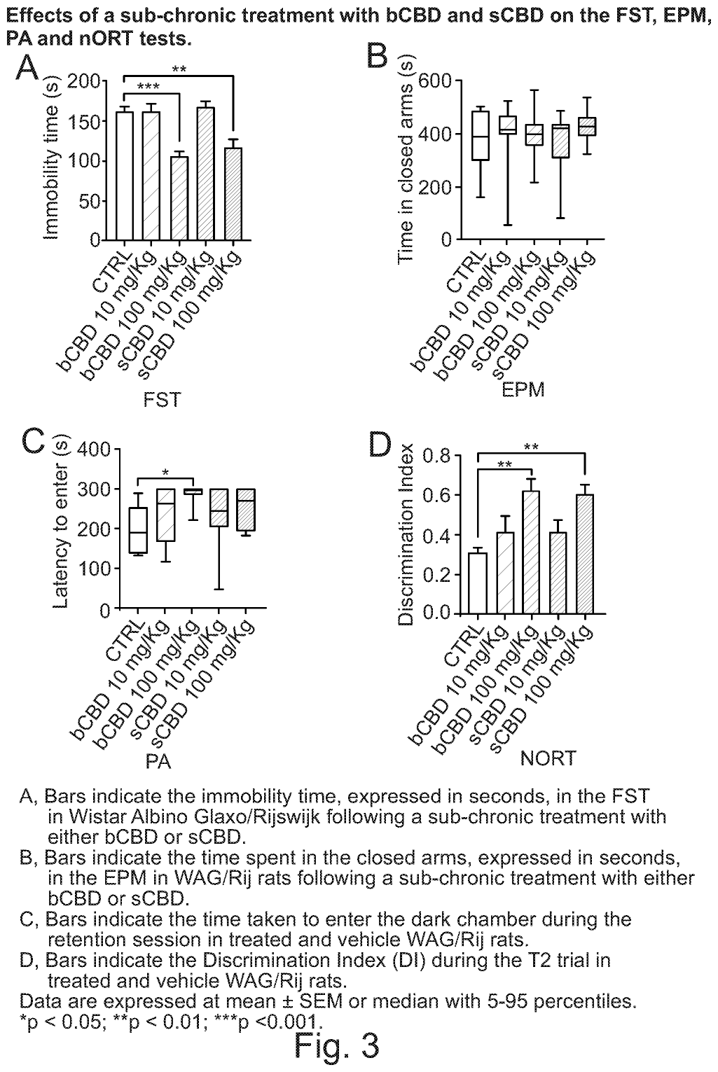

FIG. 3A shows the administration of bCBD and sCBD, in adult WAG/Rij rats, was able to significantly (F4,45=12.29; p<0.0001; n=10 per group) reduce the immobility time in comparison to control (vehicle) group.

A sub-chronic treatment with bCBD, at the higher dose used, significantly (p=0.0002) reduced the immobility time in adult WAG/Rij rats in comparison to control rats while the lower dose used did not modify the immobility time.

Similarly, a sub-chronic treatment with sCBD, at the higher dose used, was also able to induce a significant reduction (p=0.0028) of immobility time in comparison to control group while being not effective at the lower dose used.

There was no significant difference between the magnitude of bCBD and sCBD effect on immobility time at 100 mg/Kg (p >0.05). Mean velocity and total distance travelled did not significantly differ (p>0.05) between groups (data not shown).

### Elevated Plus Maze (EPM)

Anxiety-like behaviour in WAG/Rij rats was evaluated in the EPM and was not affected by either bCBD or sCBD treatments (H=2.332; p=0.67; n=10 per group) as demonstrated in FIG. 3B.

Mean velocity and total distance travelled did not significantly differ (p>0.05) among groups (data not shown).

### Passive Avoidance (PA)

Learning and memory decline has recently been confirmed in WAG/Rij rats older than 6 months.

There was no significant effect of bCBD or sCBD upon the latency (s) to enter in the dark compartment, during the conditioning trial (F4,45=1.501; p=0.38; n=10 per group) data not shown.

However, during the retention session, the treatment significantly affected the latency to enter in the dark compartment (H=9.739; p=0.0451; p=10 per group) as is shown in FIG. 3C.

The sub-chronic treatment with bCBD, at the higher dose used, significantly (p=0.0124) increased the latency to enter (s) in the dark compartment in adult WAG/Rij rats in comparison to control rats. At odds, a sub-chronic treatment either with bCBD, at the lower dose used, or with sCBD, at the two doses used, did not significantly modify (p>0.05) the latency to enter in the dark chamber.

Additionally, there was no significant difference between the effect of 100 mg/Kg bCBD when compared with 100 mg/Kg sCBD (p>0.05).

Novel Object Recognition Test (nORT)

The nORT was performed to evaluate the short-term memory in all groups, WAG/Rij rats have a poorer performance (working memory impairment) in nORT test in comparison to control rats.

FIG. 3D shows the administration of bCBD and sCBD, in adult WAG/Rij rats, was able to significantly (F4,45=6.009; p=0.0006; n=10 per group) affect the discrimination index (DI).

The sub-chronic treatment with either bCBD or sCBD, at the lower dose used, in WAG/Rij rats did not significantly (p>0.05) change DI in comparison to control rats. However, bCBD and sCBD, at 100 mg/Kg, significantly increased the DI when compared to the vehicle treated animals (p=0.0016 and p=0.0028 respectively).

No significant (p>0.05) difference in DI was detected between bCBD and sCBD treated rats at both doses.

### Three Chamber Test (TCT)

The three-chambered social task has been widely employed as a standard test for investigating sociability in rodents.

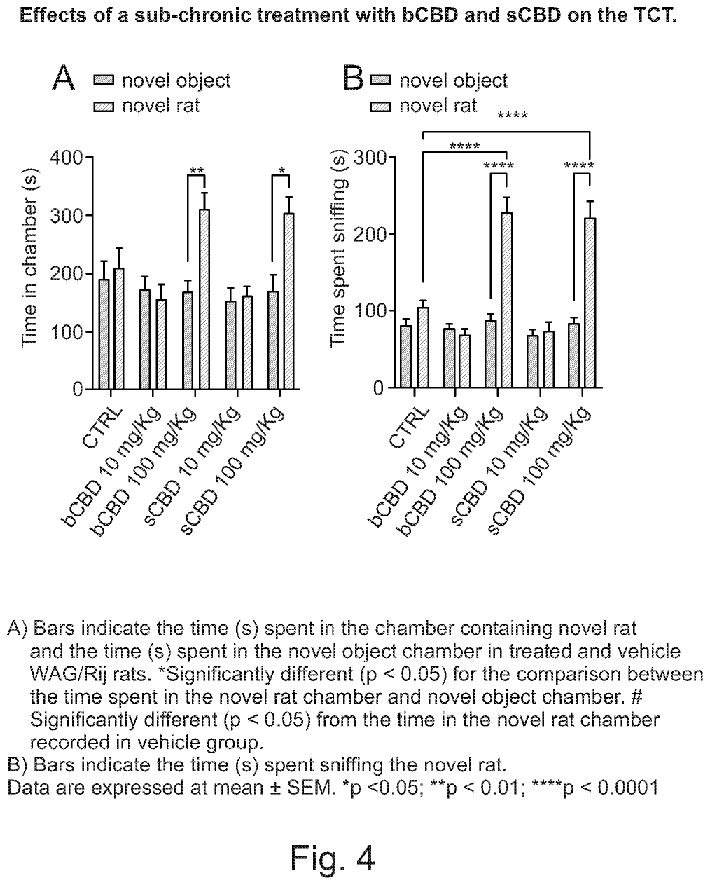

The data showed a significant interaction between the time spent in the chambers and the treatments (F4,90=4.125; p=0.0041; n=10 per group) as demonstrated in FIG. 4A.

Post-hoc analysis revealed that WAG/Rij rats after a sub-chronic treatment with either bCBD or sCBD, at 100 mg/Kg, spent significantly more time in the novel rat chamber (p=0.0089 and p=0.0173, respectively) than in the novel object chamber.

Likewise, a significant interaction between the time spent sniffing and the treatment was found (F4,90=17.15; p<0.0001; n=10 per group) as is shown in FIG. 4B.

The rats spent significantly more time sniffing the novel rat than the novel object at the 100 mg/Kg dose (p<0.0001 for both drugs), supporting an increased sociability after drug treatment.

The time spent sniffing the novel rats were significantly increased in treated WAG/Rij rats, with either bCBD or sCBD, at the higher dose, in comparison to control rats (p<0.0001 for both drugs).

Both drugs, at the lower doses used, were ineffective to significantly modify these times in WAG/Rij rats (p<0.05). Additionally, no significant (p>0.05) difference was detected between bCBD and sCBD treated rats. Furthermore, no significant (p>0.05) difference in the time spent in the centre and the number of entries in the chambers was detected among groups (data not shown).

### Rotarod Test (RT)

Treatment with sCBD and bCBD treatment did not influence (F4,45=0.1841; p=0.9455; n=10 per group) rotarod test results in any group, data not shown.

### Conclusion

These data demonstrate that botanically derived purified CBD was effective against absence seizures and this effect was accompanied by an improvement of depressive-like behaviour, memory and sociability.

CBD that was of synthetic origin was able to improve the depressive-like behaviour, memory and sociability but not the absence seizures themselves.

Such data are significant as they demonstrate that such a CBD composition may be useful in the treatment of absence epilepsy.

1. A method of treating absence epilepsy comprising administering a A cannabidiol (CBD) preparation to the subject in need thereof.

2. The method of claim 1, wherein the CBD preparation comprises greater than or equal to 98% (w/w) CBD and less than or equal to 2% (w/w) other cannabinoids, wherein the less than or equal to 2% (w/w) other cannabinoids comprise the cannabinoids tetrahydrocannabinol (THC); cannabidiol-C1 (CBD-C1); cannabidivarin (CBDV); and cannabidiol-C4 (CBD-C4), and wherein the THC is present as a mixture of trans-THC and cis-THC.

3. The method of claim 1, wherein the CBD is present is isolated from cannabis plant material.

4. The method of claim 1, wherein at least a portion of at least one of the cannabinoids present in the CBD preparation is isolated from cannabis plant material.

5. The method of claim 1, wherein the CBD is present as a synthetic preparation.

6. The method of claim 5, wherein at least a portion of at least one of the cannabinoids present in the CBD preparation is prepared synthetically.

7. The method of claim 1, wherein the dose of CBD is greater than 5 mg/kg/day.

8. The method of claim 1, wherein the dose of CBD is 20 mg/kg/day.

9. The method of claim 1, wherein the dose of CBD is 25 mg/kg/day.

10. The method of claim 1, wherein the dose of CBD is 50 mg/kg/day.

11. (canceled)
